(19) United States
(12) Patent Application Publication
(10) Pub. No.: US 2021/0379216 A1
(43) Pub. Date: Dec. 9, 2021

(54) APPARATUS AND METHODS FOR INFECTIOUS VIRUS MITIGATION

(71) Applicant: James H. Bushong, JR., Skillman, NJ (US)

(72) Inventor: James H. Bushong, JR., Skillman, NJ (US)

(21) Appl. No.: 17/012,861

(22) Filed: Sep. 4, 2020

Related U.S. Application Data
(60) Provisional application No. 63/102,257, filed on Jun. 5, 2020.
(60) Provisional application No. 63/103,539, filed on Aug. 10, 2020.

(51) Int. Cl. A61L 2/00

(52) U.S. Cl. CPC A61L 2/0082; A61L 2/0029; A61L 2/0017

(57) ABSTRACT

The present invention offers infectious virus mitigation apparatus that utilize one or more 3-dimensional porous metal substrate that impart virus mitigation effect. Fluid that contains or may contain infectious virus traverses through said substrate to achieve virus mitigation effect. Additional virus mitigation effect can be achieved by subjecting said virus mitigation apparatus to suitable wavelength(s) of light that enhance total virus mitigation effect and/or utilizing contoured cover glazing to induce fluid dynamics that can enhance total virus mitigation effect per pass of said fluid through said apparatus. The utility includes a wide variety of practical uses such as filtration of air, water, blood, and other fluids that contain or may contain infectious virus such as coronavirus.

### CROSS REFERENCE TO RELATED APPLICATIONS

This application claims the benefit of U.S. Provisional Application Ser. No. 63/102,257 filed Jun. 5, 2020 and entitled Apparatus and Methods for Infectious Virus Mitigation, and claims the benefit of U.S. Provisional Application Ser. No. 63/103,539 filed Aug. 10, 2020 and entitled Apparatus and Methods for Infectious Virus Mitigation, the entire disclosures of which are hereby incorporated by reference herein.

### FIELD OF THE INVENTION

The present invention relates generally to apparatus and methods of infectious virus mitigation. The utility of the present invention includes the practical use of filtering fluids such as air, water, blood, gases, and liquids to help mitigate infectious viruses such as those commonly referred to as coronaviruses.

### BACKGROUND OF THE INVENTION

Many common gas and liquid filters utilize various forms of cloth, woven fabrics, and plastics to physically filter undesirable materials and purify the air or water. However, it is known that the ability of certain viruses such as coronaviruses, including the current infectious virus commonly referred to as Covid-19, can survive for longer time durations on certain substrates. In particular, it has been shown that Covid-19 can survive for longer time durations on plastics and other relatively non-reactive materials such as stainless steel. In contrast, it has been shown that Covid-19 has a much lower survival time duration on copper. The survival time duration studies on various substrates (that the present inventor is aware of) were informational only, with no mechanism proposed nor hypothesis on why Covid-19 survives longer on certain substrates and decays much faster on copper. The survival time duration of infectious viruses such as Covid-19 is an important factor; anything that can shorten the survival time of such viruses would be expected to reduce the risk of infection and reduce the spread of contagious viruses such as Covid-19.

The ability to remove infectious virus such as Covid-19 via conventional physical filtration is also complicated by the extremely small size of such viruses. For example, Covid-19 virus is estimated to be between 70 and 90 nanometers in size.

The ability to remove as much infectious virus per pass in a filtration process is also highly desirable, to decrease the risk of contagion and health consequences of higher concentrations/exposure to the virus.

It has been well-documented that the most likely original host mammal of Covid-19 (SARS-CoV-2) and the previous zoonotic coronavirus (SARS-CoV-1) is bats. Bats such as the horseshoe classification of bats (Genus Rhinolophus) that have been identified as hosting coronaviruses 80+% similar in genetic code to SARS-CoV-2 and SARS-CoV-1, are largely nocturnal. Through evolution, it is normal for viruses to select hosts wherein the virus can thrive without mass-destruction of the host and without rapid destruction of the virus. SARS coronaviruses, and other coronaviruses that have evolved to thrive in the largely nocturnal host of bats, may therefore be adversely affected by constituents of sunlight such as the ultraviolet (UV) portion of the spectrum.

Bats are also known to have a very efficient circulatory system, with heart sizes that can be up to 3 times larger than an equivalent mass terrestrial mammal and heartbeat rate as high as 1,000 beats per minute. One possible reason that coronaviruses, and in particular SARS-CoV-2 and SARS-CoV-1, do not cause mass mortality in bats is that high circulatory rate prevents these viruses from taking sufficient hold to damage internal organs and tissues to the point of mortality. This is further supported by the appearance of “Covid toe” in certain otherwise healthy human individuals; circulation of blood to the toes can be among the lowest flow rates in the human body, making it a tell-tale sign that coronaviruses such as Covid-19 may take hold in areas of low flow whereas said viruses may be less efficient at taking hold in areas of high flow. Bats also maintain blood oxygen levels that are typically twice that of terrestrial mammals. These physiological factors of bats have been translated into some of the possible design-benefits of the present invention, in particular the fluid dynamic aspects.

Utilizing global satellite data and the frequency of Covid-19 cases during the early stages of the pandemic spread, an analysis by the present inventor and his son suggests that higher intensity sunlight (as measured by the UV light component) may be able to decrease the risk of exposure and spread of Covid-19.

Therefore, there is a need in the art for an apparatus and methods to mitigate viruses and other pathogens by filtering fluids though porous metal substrates, including but not limited to copper containing substrates. These and other features and advantages of the present invention will be explained and will become obvious to one skilled in the art through the summary of the invention that follows.

### SUMMARY OF THE INVENTION

The present invention relates generally to apparatus and methods for treating fluids that contain or may contain infectious viruses such as those commonly referred to as coronaviruses. Applications include air filtration, water filtration, blood filtration, and filtration of other gases and liquids that may contain infectious viruses such as coronaviruses and in particular Covid-19 and similar zoonotic coronaviruses. The practical application of the present invention is to enhance mitigation of said viruses. As used herein, the terms “mitigation” of said viruses and “mitigation effect” of said viruses refers to one or more of the following end results achieved with said apparatus; virus destruction, decrease of virus infection ability, decrease in virus activity, decrease in virus spread, decrease in the mortality of virus in humans and other mammals, decrease in the detrimental effects of the virus on humans and other mammals, and decrease in virus concentration. As used herein, the term “coronavirus” is interchangeable with singular or plural reference to any type of virus commonly referred to as “coronavirus” or “corona virus” or “coronaviruses” or “corona viruses.”

The apparatus of the present invention comprise highly porous metal substrate to aid in the mitigation of said infectious viruses. The general method of the present invention is for fluid that contains infectious virus such as coronavirus to pass through said substrate to achieve infectious virus mitigation effect, and in particular coronavirus mitigation effect. Said highly porous metal substrate are characterized by a 3-dimensional porous metal that has an open lattice-work of air pores and channels within inter-connected strands of metal, and maintains sufficient inter-connectivity of metal strands to remain a singular object. Because said substrate of the present invention comprise of substantially inter-connected strands of said metal, the porosity of said substrate of the present invention can be higher while still retaining strength to be a singular object compared to individual strands of materials that are woven or pressed together. The porosity of said substrate can range from approximately 85% to 98%, or from approximately 90% to about 98%. The porosity of the metal substrate for the present invention can be selected such that fluids such as air, water, blood, and other gases and liquids that contain or are suspected to contain infectious virus such as coronavirus can be passed directly throughout any of the length, depth, and width axis of the substrate. The air pores located in-between the substantially inter-connected strands of metal form air channels that are substantially inter-connected throughout the length, width, and depth axis of the porous metal substrate of the present invention so that said fluids can traverse throughout any of the length, width and depth axis of said substrate.

The porosity of the metal substrate of the present invention can be selected such that said fluid can pass directly through path lengths of the porous 3-dimensional substrate, such as for example 3 millimeters (mm) path lengths or more, or 10 mm path lengths or more, or 100 mm path lengths or more, or even 500 mm path lengths or more in a single-pass through. The long path length for said fluid to traverse while contacting the high exposed surface area of inter-connected strands of metal in the porous 3-dimensional substrate enables excellent opportunity for virus to contact the copper strands as the fluid in which the virus is present traverses through said substrate, thereby offering efficient virus mitigation effect.

Said highly porous substrate of the present invention can also enable the opportunity for said fluid to contact the substrate at higher flow rates compared to lower porosity substrates commonly employed in conventional filtration apparatus. Higher flow rates can lead to higher velocity and greater force of impact between the virus matter and the metal surfaces of the present invention; this may be beneficial to virus mitigation in an analogous manner to the potential benefits of a high efficiency circulatory system of bats, and the telltale signs that coronaviruses seem to thrive in low-flow environments as indicated by phenomena such as “Covid toe.” A conventional physical filtration apparatus would require a filtration rating of approximately 70 to 90 nanometers (the approximate size of for example Covid-19 virus particles)—a size so small that it would be difficult or impossible to achieve the flow rate capabilities of the present apparatus that have a substrate pore size of 0.1 mm or greater or even 1 mm or greater or even 3 mm or greater.

Said porous substrate of the present invention can also create a more tortuous fluid path within the 3-dimensional metal matrix compared to conventional filtration apparatus; said increased tortuous fluid path may increase the shear on infectious virus that are susceptible to mitigation based on shear; coronavirus are among infectious virus known to have a mitigation effect upon the virus via shear. The combination of higher flow rates and increased tortuous fluid path in the porous substrate of the present invention can be particularly advantageous for mitigating coronavirus compared to conventional filtration apparatus and methods.

If said fluid is air, blood, or other oxygen-containing fluids, higher flow rates may be beneficial to achieve higher net oxygen molecules in contact with the virus; this may be beneficial for virus mitigation in an analogous manner to the potential benefits of high oxygen levels in bats. The ability to enable higher flow rates also increases the possibilities for supplementing the fluid flow with supplemental oxygen to achieve similar potential virus mitigation benefits.

The metal of the porous substrate of the present invention can be selected from pure copper of approximately 98% purity or greater copper (Cu), copper oxides such as CuO or Cu₂O, and any combination of pure copper and copper oxides. The coronaviruses SARS-CoV-2 and SARS-CoV-1 have been shown to have a considerably faster decay rate on pure copper compared to for example stainless steel and plastic. Copper oxides may provide an oxygen-related mitigation effect, based on the analogy to the potential benefit of high oxygen levels found within bats. Copper oxides may offer a catalytic mitigation process in which the copper in the copper oxide becomes reduced while oxidizing one or more portions of for example a coronavirus to cause a mitigation effect. As long as some oxygen is present in the virus-containing fluid passing through the porous metal substrate (or a supplemental flow of oxygen is added), the reduced copper may be re-oxidized to the copper oxide state for continued catalytic mitigation of for example a coronavirus. Other inorganic copper compounds and organometallic copper compounds that have infectious virus mitigation effect are contemplated for use in the present invention. Alloys of copper and zinc, and/or copper oxide and/or zinc oxide, at any ratio between copper, zinc, copper oxide, and zinc oxide that have infectious virus mitigation effect, are also contemplated for use in the present invention. Other metals, metal oxides, metal alloys, metalloids, organic compounds, or organometallic compounds that have infectious virus mitigation effect and that can be configured according to the apparatus and methods of the present invention are also contemplated. Materials with infectious virus mitigation effect can be incorporated into or coated onto the porous metal substrate of the present invention.

The pore size of the porous metal substrate of the present invention can range from about 0.1 mm to about 10 mm, or from about 0.1 to about 5 mm, or from about 0.1 to about 3 mm. The volume density of the porous metal substrate of the present invention can be from approximately 0.1 to about 3 grams per cubic centimeter (cc), or from about 0.1 to about 2 grams/cc, or from about 0.1 to about 1 grams/cc. The porous metal substrate of the present invention is a 3-dimensional structure, with a depth of the porous metal substrate that can range from about 1 mm to about 100 mm, or from about 1 mm to about 50 mm, or from about 1 mm to about 25 mm, or from about 1 to about 10 mm, or from about 2 to about 10 mm, or from about 3 to about 6 mm.

Open-celled foamed metal structures can be utilized for the present invention, wherein open-cell foamed metal structures comprise of air pores that form air channels that are substantially inter-connected throughout the length, width, and depth axis of the porous metal substrate, and wherein these open-celled foamed metal structures maintain exceptionally high porosity while possessing sufficient inter-connected strands of metal to remain sufficiently strong as to remain a singular object.

The porous metal substrate of the present invention may be encased in cover glazing (housing) to form a complete seal around the porous metal substrate. One or more entry ports and one or more exit ports can be incorporated into any sidewall or glazing face in order to enable fluid to enter and exit said substrate. If said substrate is subjected to a light source of suitable wavelength(s) with infectious virus mitigation effects, said light source may be housed inside said cover glazing, or said cover glazing may be such that an external source of said suitable wavelength(s) can penetrate said cover glazing.

The apparatus of the present invention may further comprise a flow-pattern configuration to offer increased net path length per pass between infectious virus containing fluid and said porous metal substrate of the present invention. For example, the present invention can feature cover glazings in contoured shapes that guide an infectious virus containing fluid through the porous metal substrate in a specific manner, such that the fluid (and virus contained therein) can efficiently contact a longer path length of porous metal per fluid pass. For mitigation of constituents such as coronavirus, it can be beneficial to mitigate as much of said constituents as possible in a single pass. The flow pattern induced by said cover glazings through the porous metal substrate and apparatus of the present invention can enable greater mitigation of infectious virus per pass-through of said apparatus, and thereby decrease the risk of infectious virus contagion between said virus and for example people. Said flow pattern may also increase the velocity of fluid flow at the points of contact between said infectious virus containing fluid and said porous metal substrate; said increased fluid velocity may for example increase shearing forces between said fluid and said porous metal substrate, and thereby increase the shear on one or more portions of the infectious virus and provide an additional virus mitigation effect.

The specific manner in which for example said contoured-shaped cover glazings guide said fluid is such that said fluid is guided along the top, through the depth, and along the bottom of said porous metal substrate at alternating intervals along at least the length axis of said porous metal substrate. As used herein, the term “alternating intervals” means that the fluid flow switches from being substantially on the top and through the depth of said porous metal substrate along some distance of the length axis, to being substantially on the bottom and through the depth of said porous metal substrate along some distance of the length axis of said porous metal substrate. As used herein, the length axis of said porous metal substrate is referred to as the “Y axis” of the porous metal substrate, and represents the axis corresponding to the net direction of fluid flow along said substrate. As used herein, the width axis of said porous metal substrate is referred to as the “X axis.” Said fluid is guided through the depth axis of said porous metal substrate by having for example the contour of said cover glazing guide said fluid flow through the depth axis of said porous metal substrate at alternating intervals along at least the Y axis. As used herein, the depth axis of the porous metal substrate is referred to as the “Z axis.” This method of alternating interval, contoured flow along the Y axis of said porous substrate can enable a substantially longer net path length and contact exposure of said fluid through said substrate per pass by guiding the fluid to pass through the Z axis multiple times in a single pass through the apparatus of the present invention. For comparison, a typical filtration process flow wherein the fluid flows orthogonally to the XY plane of a substrate would pass through the Z axis only once per pass. When used in the present invention, the contours of said cover glazings can for example exhibit alternating geometric patterns across any certain location of the Y and/or X axis of said porous metal substrate in order to induce an alternating “top and bottom” flow of the fluid across at least the length axis of the porous metal substrate.

The porous metal substrate of the present invention can be subject to a light source that features one or more wavelengths of light with infectious virus mitigation effects, and in particular coronavirus mitigation effects. For example, subjecting a porous copper metal substrate of the present invention with UV light may have additional virus mitigation effects, and in particular may have additional virus mitigation effects on coronaviruses such as SARS-CoV-2 and SARS-CoV-1 and other viruses that have naturally evolved in nocturnal mammals such as bats. In addition to UV light, additional wavelengths of light within the electromagnetic spectrum may have coronavirus mitigation effects, particularly for coronaviruses and other viruses that have naturally evolved in nocturnal mammal species such as bats. In the present invention, any wavelength of light with infectious virus mitigation effects may be utilized in conjunction with said substrate. The high porosity of the metal substrate of the present invention can allow said light to penetrate throughout the depth of said substrate at a considerably greater depth than lower porosity substrates often employed in filtration apparatus. Said highly porous metal substrate of the present invention offers exceptional reflection and refraction of said light within said substrate vast pore structures, and this can enable more of the surface area of said substrate to be subjected to said light. Wavelength(s) of light with infectious virus mitigation effect can therefore be of greater effective utilization throughout the apparatus of the present invention, and can provide an effective dual-mitigation effect when combined with the infectious virus mitigation effects of the porous metal substrate.

Fluid containing infectious virus can enter into the infectious virus mitigation apparatus of the present invention, either by means of forced convection by pulling vacuum at the outlet of the infectious virus mitigation apparatus with a mechanical device such as an air blower or air compressor or pump, or by forced convection by pressurizing a fluid with a mechanical device such as an air blower or air compressor or pump at an inlet of the infectious virus mitigation apparatus, or by gravity, or natural convection, or by thermosiphoning, or any other means of fluid motion. Said fluid and said virus therein that traverse via convection throughout the 3-dimensional porous metal substrate can efficiently contact the porous metal substrate of the present invention to achieve efficient infectious virus mitigation effect.

Examples of useful applications of the present invention include apparatus for the filtration of air to achieve Covid-19 mitigation effects on airplanes, in hospitals, in office buildings, in manufacturing facilities, in hotels, restaurants, cruise ships, and the like wherein large air handler systems process air for a large number of people in for example HVAC applications. Effective mitigation of Covid-19 in such locations can help economies return to normal operations with lower risk of future outbreaks.

Another example of useful application of the present invention is for use in home filtration systems, such as air filters utilized in HVAC applications.

Another example of possible useful application of the present invention include apparatus for filtering the blood of patients infected with coronavirus. Patients diagnosed with coronavirus can have their blood recirculated through the virus mitigation apparatus of the present invention in a dialysis-style treatment process. The present invention can serve as a stand-alone dialysis style process for coronavirus mitigation for example, or be utilized in conjunction with conventional dialysis as a further means to purify the blood of individuals suffering from kidney disorders, acute kidney infections, and related diseases for example. People with pre-existing kidney disorders have exhibited among the highest co-morbidity rates when infected with Covid-19.

Another example of possible useful application can be for the treatment of potable water wherein infectious virus may be present.

Further, since the porosity of the porous metal substrate of the present invention can be as high as 95 to 98% for the infectious virus mitigation apparatus of the present invention, these advantages and utility are achieved with very little metal mass. Conserving metal mass while still obtaining outstanding performance advantages is a key breakthrough for widespread availability of the apparatus of the present invention.

### BRIEF DESCRIPTION OF DRAWINGS

FIG. 1 shows a top and side view of an example of the 3-dimensional porous metal substrate of the present invention, with Reference numeral 1 pointing to an example of strand of metal that is substantially inter-connected to other strand of metal such that the inter-connected strands of metal form the singular 3-dimensional object indicated as Reference 3, with an open lattice-work of air pores such as shown by Reference 2 in-between the substantially inter-connected strands of metal, with depth of the 3-dimensional porous metal substrate indicated by Reference 4, length indicated by Reference 5, and width indicated by Reference 6.

FIG. 2 shows an example of the axis conventions for the 3-dimensional porous metal substrate, cover glazing, and apparatus of the present invention, wherein Reference 7 refers to a width axis, shown as the “X axis” of the cover glazing, porous metal substrate, and apparatus of the present invention, Reference 9 refers to a length axis and corresponds to the net direction of fluid flow into and out of the apparatus, shown as the “Y axis” of the cover glazing, porous metal substrate, and apparatus, and Reference 8 refers to the depth axis, shown as the “Z axis” of the cover glazing, porous metal substrate, and apparatus of the present invention. In FIG. 2, the plane of the paper is identical to the plane of the X and Y axis, with the Z axis going “into” the paper.

FIG. 3 shows a side view of a cross-sectional slice of an example of an infectious virus mitigation apparatus of the present invention, with an example of the porous metal substrate enclosed in an example of the contoured cover glazings to induce alternating flow patterns of infectious-virus containing fluid along the length axis of the porous metal substrate, with depth of the porous metal substrate along the “Z axis” and length along the “Y axis”, wherein Reference 10 refers to an entrance for fluid containing infectious virus into the space between a contoured top cover glazing and the porous metal substrate, Reference 11 refers to fluid also having the ability of entering through the depth of the porous metal substrate, Reference 12 refers to a decreasing contour line of the top cover glazing to begin to guide the fluid through the depth of the porous metal substrate, Reference 13 refers to a flat area of the bottom cover glazing that is close to or even touching the porous metal substrate to substantially restrict most of the flow from passing under the porous metal substrate, and Reference 14 refers to an increasing contour line along the bottom cover glazing to substantially allow the fluid to enter along the bottom of the porous metal substrate, and Reference 15 refers to a flat area of the top cover glazing that is close to or even touching the porous metal substrate to substantially restrict most of the flow from passing on top of the porous metal substrate, and Reference 16 refers to a decreasing contour line along the bottom cover glazing to begin to guide the fluid through the depth of the porous metal substrate, and Reference 17 refers to an increasing contour line along the top cover glazing to substantially allow the fluid to enter along the top of the porous metal substrate, and Reference 18 refers to an increasing contour line along the bottom cover glazing to substantially allow fluid to enter along the bottom of the porous metal substrate and to exit this example of an infectious virus mitigation apparatus of the present invention, and Reference 19 refers to fluid also having the ability to exit out the depth of the porous metal substrate, and Reference 20 refers to a light source that emits one or more portions of the electromagnetic spectrum that can reflect and refract throughout said porous metal substrate to achieve additional virus mitigation effect.

FIG. 4 shows a side view of a cross-sectional slice of an example of a contoured cover glazing of the apparatus of the present invention but without the porous metal substrate shown. Reference 21 refers to the top cover glazing, Reference 22 refers to the bottom cover glazing, and Reference 20 refers to a light source that emits one or more portions of the electromagnetic spectrum that can reflect and refract throughout said porous metal substrate to achieve additional virus mitigation effect.

FIG. 5 shows a side view of a cross-sectional slice of an example of a infectious virus mitigation apparatus of the present invention, with identical concepts and Reference numbers as FIG. 3 but with a different shape of contours to demonstrate just one of many other example of contour dimensions and shapes that can be utilized to achieve the alternating air flow of substantially on top of and through the depth of the porous metal substrate to on the bottom of and through the depth of the porous metal substrate along at least the length axis of the porous metal substrate.

FIG. 6 shows a side view of a cross-sectional slice of an example of a contoured cover glazing of the apparatus of the present invention, with flow lines showing the basic idea of guiding of the fluid through the porous metal substrate in a manner as described in FIG. 5, but without the porous metal substrate shown. Reference 21 refers to the top cover glazing, Reference 22 refers to the bottom cover glazing, Reference 23 refers to fluid entering the infectious virus mitigation of the present invention and being guided by the contoured cover glazings in the general direction of flow shown by the arrows throughout the length axis of the porous metal substrate, and fluid exiting the infectious virus mitigation shown by Reference 24, and Reference 20 refers to a light source that emits one or more portions of the electromagnetic spectrum that can reflect and refract throughout said porous metal substrate to achieve additional virus mitigation effect and is located either internally or externally to the apparatus of the present invention.

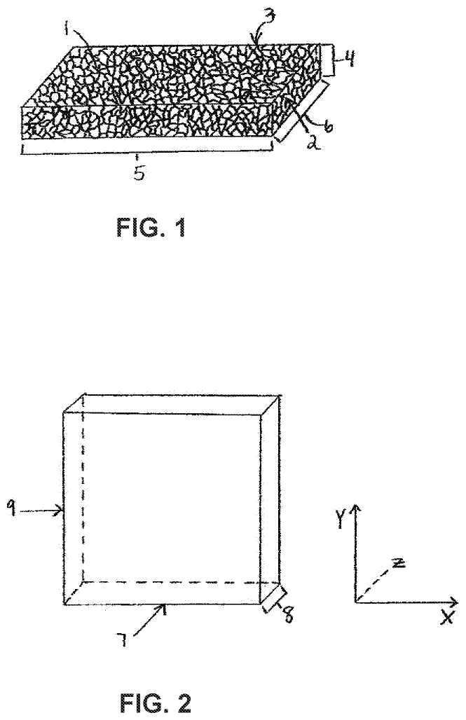

Further novel features and other advantages of the present invention will become apparent from the following description, discussion, and the appended claims.

### DETAILED DESCRIPTION

Although specific embodiments of the present invention will now be described, it should be understood that such embodiments are by way of example only and merely illustrative of but a small number of the many possible specific embodiments which can represent applications of the principles of the present invention. Changes and modifications by persons skilled in the art to which the present invention pertains are within the spirit, scope and contemplation of the present invention as further defined in the appended claims. All references cited herein are incorporated by reference as if each had been individually incorporated.

In one embodiment of the present invention, copper foam of about 98% purity or greater, porosity between about 85 and 98%, pore size between about 0.1 and 10 mm, density between about 0.1 to 3 grams/cubic centimeter, and depth between 1 and 1000 mm is utilized as the porous metal substrate for infectious virus mitigation. Said porous metal substrate is housed in cover glazing with an inlet and outlet to form an infectious virus mitigation apparatus of the present invention. Fluid that requires purification to mitigate risk of for example Covid-19 virus or other coronavirus is passed through said apparatus via convection. With one or more passes of said fluid through said apparatus, said virus undergoes one or more virus mitigation effects.

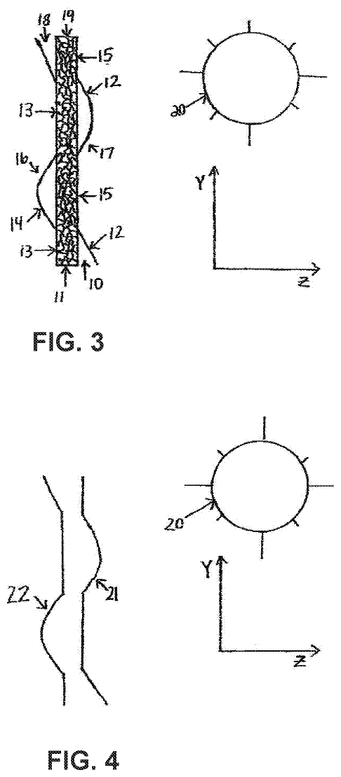

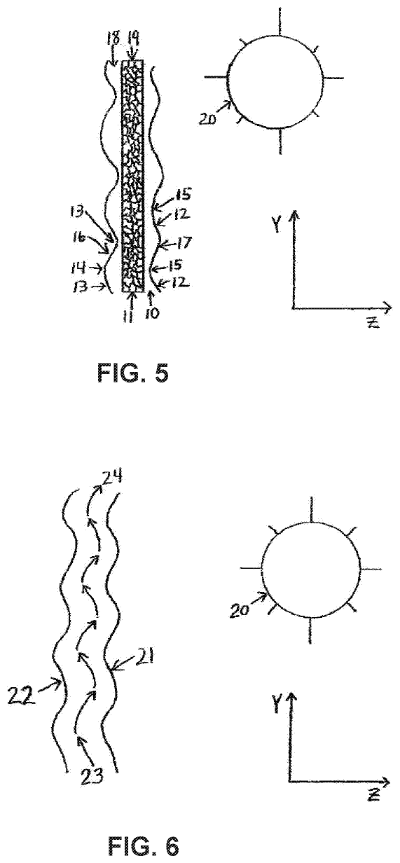

In another embodiment of the present invention, copper foam of about 98% purity or greater, porosity between about 85 and 98%, pore size between about 0.1 and 10 mm, density between about 0.1 to 3 grams/cubic centimeter, and depth between 1 and 1000 mm is utilized as the porous metal substrate for infectious virus mitigation, and said porous metal substrate is housed in cover glazing with an inlet and outlet, and wherein said cover glazings comprise alternating geometric contours such as those shown in FIGS. 3, 4, 5, and 6 to comprise an infectious virus mitigation apparatus. Fluid that requires purification to mitigate risk of for example Covid-19 virus or other coronavirus is passed through said apparatus via convection, and said fluid is guided by said contoured cover glazing such that said fluid traverses along the top, through the depth, and along the bottom of said porous metal substrate at alternating intervals along the net direction of fluid flow through said apparatus With one or more passes of said fluid through said apparatus, said virus undergoes one or more virus mitigation effects.

In other embodiments, any of the embodiments are further subject to UV light. The method of adding UV light to the process may increase the total virus mitigation effect for these embodiments of the present invention.

In other embodiments, any of the embodiments are further subject to any wavelength(s) of the electromagnetic spectrum that are found to increase total virus mitigation effect.

In other embodiment, any of the embodiments are placed outdoors and subject to sunlight. One or more wavelengths of light in the solar spectrum may increase the total virus mitigation effect for these embodiments of the present invention. These embodiments can be particularly useful for mitigating the risk of corona viruses such as Covid-19 in large air handler systems such as those present in office buildings, manufacturing facilities, and warehouses. This method can have a further advantage of harvesting solar thermal energy during winter months, as the porous copper makes a highly efficient solar thermal energy collector as detailed in U.S. Pat. No. 9,318,996 by the inventor of the present invention.

In other embodiments, any of the embodiments are a medical device that is used to filter bodily fluids such as blood to achieve infectious virus mitigation effect.

In another embodiment of the present invention, the metal of the porous metal substrate is copper oxide. The copper oxide may impart a form of oxidation to said Covid-19 virus and achieve a virus mitigation effect, while the copper oxide is reduced to copper (Cu) or a lower oxidation state of copper oxide (for example, CuO being reduced to Cu₂O). The oxygen in for example air or blood passing through the apparatus may re-oxidize the copper metal to copper oxide (or Cu₂O to the higher oxidation state CuO), rejuvenating the substrate to the potentially higher reactive state (relative to a virus mitigation effect reaction with coronavirus for example) of copper oxide. Oxygen-enriched air or blood, containing oxygen levels higher than ambient air or blood, may for example also be utilized in rejuvenation cycles to re-oxidize the Cu or Cu₂O at a faster rate or when deemed beneficial for any reason.

In other embodiments, two or more different materials are incorporated into the apparatus—as long as one or more of said materials comprise the porous metal substrate with virus mitigation effect of the present invention. Examples include 2 different materials incorporated into the metal strands that form the inter-connected strands of metal of the porous metal substrate, or a coating of a different material with virus mitigation effect onto the porous metal substrate, a pre-filter to remove particulates, or 2 or more porous metal substrates comprising different metals, metal alloys, metal oxides, organometallics, or organic substances with virus mitigation effect.

In other embodiments, more than one porous metal substrate with infectious virus mitigation effect can be utilized in the same infectious virus mitigation apparatus of the present invention, either placed side by side to increase either or both of the total length and width of porous metal substrate or placed on top of one another to increase the total depth of porous metal substrate. Other substrates, such as those that can pre-filter non-virus particulates, can be utilized in conjunction with the apparatus of the present invention.

In other embodiments, any combination of the above embodiments can be utilized together.

I claim:

1. A coronavirus mitigation apparatus comprising of:
a substrate, said substrate comprises porous metal, in which said porous metal comprises substantially inter-connected strands of metal, said porous metal further comprises a substantially open lattice work of air pores between said substantially inter-connected strands of metal, wherein said inter-connected strands of metal being configured to form a substantially singular object with said open lattice work of air pores, in which said porous metal further comprises a porosity of approximately 85% to 98%, and said open lattice work of air pores comprises a pore size of approximately 0.1 mm to 10 mm, said porous metal further comprises a volume density of approximately 0.1 to 3 grams per cubic centimeter, wherein said porosity, pore size, and density of said porous metal being configured to enable a fluid to substantially traverse throughout said open lattice work of air pores between said inter-connected strands of metal of said substrate, and wherein said fluid contains or may contain one or more coronavirus, and said porous metal substrate further comprises a depth of approximately 1 mm to 1000 mm, and wherein said fluid containing said coronavirus undergoes an infectious virus mitigation effect as it traverses through said substrate.

2. The coronavirus mitigation apparatus of claim 1, in which said infectious virus mitigation effect comprises one or more of: virus destruction, decrease of virus infection ability, decrease in virus activity, decrease in virus spread, decrease in mortality of virus in humans and other mammals, decrease in detrimental effects of virus on humans and other mammals, and decrease in virus concentration.

3. The coronavirus mitigation apparatus of claim 1, in which said substantially inter-connected strands of metal of said porous metal substrate substantially comprises one or more materials with said infectious virus mitigation effect selected from a group comprising copper, zinc, copper oxides, zinc oxides, periodic table of elements metals, periodic table of elements metalloids, metal oxides, organometallic compounds, and any other metal or metal compound that can achieve said infectious virus mitigation effect in said fluid traversing through said coronavirus mitigation apparatus, and any combinations of metals or metal compounds thereof.

4. The coronavirus mitigation apparatus of claim 1 in which an organic compound with said infectious virus mitigation effect is coated in part or in whole onto said porous metal substrate while still retaining said porosity.

5. The coronavirus mitigation apparatus of claim 1, in which said porous metal substrate is open-celled foamed metal.

6. The coronavirus mitigation apparatus of claim 1, wherein said coronavirus mitigation apparatus forms at least part of a filtration apparatus for filtering fluid that contains or may contain said coronavirus, and wherein said fluid is selected from the list of air, water, blood, gas, or liquid.

7. The coronavirus mitigation apparatus of claim 1, wherein said coronavirus mitigation apparatus is subjected to a light source emitting one or more wavelengths of light, and wherein said wavelengths of light have an additive infectious virus mitigation effect.

8. The coronavirus mitigation apparatus of claim 1, wherein shear from fluid dynamic effects provide an additive infectious virus mitigation effect.

9. An infectious virus mitigation apparatus comprising of:
a substrate, said substrate comprises porous metal, in which said porous metal comprises substantially inter-connected strands of metal, said porous metal further comprises a substantially open lattice work of air pores between said substantially inter-connected strands of metal, wherein said inter-connected strands of metal being configured to form a substantially singular object with said open lattice work of air pores, in which said porous metal further comprises a porosity of approximately 85% to 98%, and said open lattice work of air pores comprises a pore size of approximately 0.1 mm to 10 mm, said porous metal further comprises a volume density of approximately 0.1 to 3 grams per cubic centimeter, wherein said porosity, pore size, and density of said porous metal being configured to enable a fluid to substantially traverse throughout said open lattice work of air pores between said inter-connected strands of metal of said substrate, and wherein said fluid contains or may contain one or more infectious virus, and said porous metal substrate further comprises a depth of approximately 1 mm to 1000 mm, and wherein said fluid containing said infectious virus undergoes an infectious virus mitigation effect as it traverses through said substrate, and said porous metal substrate is subjected to a light source emitting one or more wavelengths of light, and wherein said wavelengths of light have an additive infectious virus mitigation effect to the porous metal substrate.

10. The infectious virus mitigation apparatus of claim 9, in which said infectious virus mitigation effect comprises one or more of: virus destruction, decrease of virus infection ability, decrease in virus activity, decrease in virus spread, decrease in mortality of virus in humans and other mammals, decrease in detrimental effects of virus on humans and other mammals, and decrease in virus concentration.

11. The infectious virus mitigation apparatus of claim 9, in which said substantially inter-connected strands of metal of said porous metal substrate substantially comprises one or more materials with said infectious virus mitigation effect selected from the group comprising copper, zinc, copper oxides, zinc oxides, periodic table of elements metals, periodic table of elements metalloids, metal oxides, organometallic compounds, and any other metal or metal compound that can achieve said infectious virus mitigation effect in a fluid traversing through said infectious virus mitigation apparatus, and any combinations of metals or metal compounds thereof.

12. The infectious virus mitigation apparatus of claim 9 in which an organic compound with said infectious virus mitigation effect is coated in part or in whole onto said porous metal substrate while still retaining said porosity.

13. The infectious virus mitigation apparatus of claim 9, in which said porous metal substrate is open-celled foamed metal.

14. The infectious virus mitigation apparatus of claim 9 wherein said light source is sun light, and wherein said apparatus is also utilized to harvest solar energy.

15. The infectious virus mitigation apparatus of claim 9, wherein the infectious virus mitigation apparatus forms at least part of a filtration apparatus for filtering fluid that contains or may contain said infectious virus, and wherein said fluid is selected from the list of air, water, blood, gas, or liquid.

16. The infectious virus mitigation apparatus of claim 9, wherein said infectious virus is one or more coronavirus.

17. The infectious virus mitigation apparatus of claim 9, wherein shear from fluid dynamic effects provide an additive infectious virus mitigation effect.

18. An infectious virus mitigation apparatus comprising of:
a substrate, said substrate comprises porous metal, in which said porous metal comprises substantially inter-connected strands of metal, said porous metal further comprises a substantially open lattice work of air pores between said substantially inter-connected strands of metal, wherein said inter-connected strands of metal being configured to form a substantially singular object with said open lattice work of air pores, in which said porous metal further comprises a porosity of approximately 85% to 98%, and said open lattice work of air pores comprises a pore size of approximately 0.1 mm to 10 mm, said porous metal further comprises a volume density of approximately 0.1 to 3 grams per cubic centimeter, wherein said porosity, pore size, and density of said porous metal being configured to enable a fluid to substantially traverse throughout said open lattice work of air pores between said inter-connected strands of metal of said substrate, and wherein said fluid contains or may contain one or more infectious virus, and said porous metal substrate further comprises a depth of approximately 1 mm to 1000 mm, and said infectious virus mitigation apparatus further comprises contoured cover glazing, in which said contoured cover glazing enclose said substrate, and wherein said contoured cover glazing comprises at least one entrance and one exit for a fluid to enter and exit said infectious virus mitigation apparatus, and wherein said contoured cover glazing being configured to guide a fluid in an alternating fashion of fluid flow substantially on a top of and through the depth of said substrate to substantially on a bottom of and through the depth of said substrate along at least one axis of said substrate selected from a length axis and a width axis, and wherein said fluid containing said infectious virus undergoes an infectious virus mitigation effect as it traverses through said apparatus.

19. The infectious virus mitigation apparatus of claim 18, in which said infectious virus mitigation effect comprises one or more of: virus destruction, decrease of virus infection ability, decrease in virus activity, decrease in virus spread, decrease in mortality of virus in humans and other mammals, decrease in detrimental effects of virus on humans and other mammals, and decrease in virus concentration.

20. The infectious virus mitigation apparatus of claim 18, in which said substantially inter-connected strands of metal of said porous metal substrate substantially comprises one or more materials with said infectious virus mitigation effect selected from the group comprising copper, zinc, copper oxides, zinc oxides, periodic table of elements metals, periodic table of elements metalloids, metal oxides, organometallic compounds, and any other metal or metal compound that can achieve said infectious virus mitigation effect in a fluid traversing through said infectious virus mitigation apparatus, and any combinations of metals or metal compounds thereof.

21. The infectious virus mitigation apparatus of claim 18 in which an organic compound with said infectious virus mitigation effect is coated in part or in whole onto said porous metal substrate while still retaining said porosity.

22. The infectious virus mitigation apparatus of claim 18, in which said porous metal substrate is open-celled foamed metal.

23. The infectious virus mitigation apparatus of claim 18, wherein said infectious virus mitigation apparatus forms at least part of a filtration apparatus for filtering fluid that contains or may contain said infectious virus, and wherein said fluid is selected from the list of air, water, blood, gas, or liquid.

24. The infectious virus mitigation apparatus of claim 18, wherein said infectious virus is one or more coronavirus.

25. The infectious virus mitigation apparatus of claim 18, wherein said infectious virus apparatus is subjected to a light source emitting one or more wavelengths of light, and wherein said wavelengths of light have an additive infectious virus mitigation effect.

26. The infectious virus mitigation apparatus of claim 18, wherein shear from fluid dynamic effects provide an additive infectious virus mitigation effect.
